(19) United States
(12) Patent Application Publication
(10) Pub. No.: US 2003/0175845 A1
(43) Pub. Date: Sep. 18, 2003

(54) Use of sulfitolysis in high performance peptide mapping

(76) Inventors: Suresh M. Kalbag, Cupertino, CA (US); Chulani Karunatilake, Danville, CA (US)

Correspondence Address: HELLER EHRMAN WHITE & MCAULIFFE LLP, 275 MIDDLEFIELD ROAD, MENLO PARK, CA, 94025-3506, US

(21) Appl. No.: 10/387,645

(22) Filed: Mar. 12, 2003

Related U.S. Application Data
(60) Provisional application No. 60/364,992, filed on Mar. 13, 2002.

(51) Int. Cl. C12Q 1/37

(52) U.S. Cl. 435/23

(57) ABSTRACT

The present invention relates to a method for high performance peptide mapping of a polypeptide with one or more cysteine residues by subjecting the polypeptide to sulfitolysis in the peptide mapping procedure.

### CROSS-REFERENCE TO RELATED APPLICATIONS

This is a non-provisional application filed under 37CFR 1.53(b), claiming priority under USC Section 119(e) to provisional Application Ser. No. 60/364,992 filed on Mar. 13, 2002.

### BACKGROUND OF THE INVENTION

1. Field of the Invention

The present invention is related to peptide mapping. More specifically, it is related to characterization and quality control of recombinant protein pharmaceuticals.

2. Description of the Related Art

Peptide mapping is an important technique used in the characterization and quality control of recombinant protein pharmaceuticals (Hancock 1995, Gamick 1992, Hoff et al. 1996, Dougherty et al. 1990). The technique is initially used during product development to verify the primary amino acid sequence and subsequently to monitor the batch-to-batch consistency of the manufacturing process. In the quality control laboratory, the peptide map is also used to confirm the identity of the protein in comparison to an extensively characterized reference material (Kannan et al. 1997). The multistep procedure involves unfolding of the large protein, reduction of disulfide bonds, capping of sulfhydryl groups, proteolytic digestion and reversed-phase high performance liquid chromatography (HPLC). Some effort has been extended toward automation of this laborious process, mostly directed toward on-line digestion using immobilized enzyme cartridges and column switching (Nadler et al. 1996). However, the sample preparation steps have remained essentially unchanged over the years.

The reduction of disulfide bonds and the blocking of sulfhydryl groups are necessary for most larger proteins in order to unfold the molecule completely, so that efficient proteolytic digestion can occur. The currently used procedure involves reduction of disulfide bonds with dithiothreitol (DTT) and alkylation of sulihydryl groups with iodoacetic acid (IAA), iodoacetamide or 4-vinylpyridine. This procedure is lengthy and cumbersome to perform because of several reagents that need to be freshly prepared and the light sensitivity of the alkylating reagent.

Oxidative sulfitolysis is a mild disulfide cleavage reaction, generating thiol-labile S-sulfonate groups at cysteines in a protein, which can be performed, for example, according to the method described, for example, by R. C. Marshall and A. S. Inglis in “Practical Protein Chemistry—A Handbook” (Publisher A. Darbre) 1986, pages 49-53 or in U.S. Pat. No. 4,923,967. Sulfitolysis has been successfully used in the refolding of E. coli-expressed proteins proinsulin (Heath et al. 1992) and insulin-like growth factor-I (Belagaje et al. 1997). There are several examples of the use of sulfitolysis for structure elucidation of proteins such as fibrinogen (Cartwright et al. 1971), immunoglobulins (Novotny et al. 1970) and ribonuclease (Milburn et al. 1988).

Antibodies are by far the most abundant among the various recombinant biopharmaceuticals that have recently received regulatory approval or are in late-stage clinical testing. Trastuzumab (rhuMAb HER2) is a (humanized) recombinant IgG1-subclass antibody that binds to the extracellular region of the human epidermal growth factor receptor 2 tyrosine kinase (Carter et al. 2000), recently approved with the trade name Herceptin® for the treatment of metastatic breast cancer in patients (Cohen 1999, Carter et al. 2000) that overexpress HER2. Reproducible peptide mapping of monoclonal antibodies is challenging due to the large molecular mass (150,000 Da, 60+ tryptic peptides), multimeric nature (2 heavy chains and 2 light chains), extensive disulfide bonding (16 per molecule) and post-translational modifications such as glycosylation (Parekh 1994) and C-terminal processing (Rao et al. 1991, Harris et al. 1993, Harris 1995).

Thus, in the rapidly growing field of recombinant protein pharmaceuticals there is a need for a more efficient method in peptide mapping which results in saving significant amounts of time and uses stable and less toxic chemicals compared to the traditional reduction/alkylation methods, all while meeting ICH guidelines (International Conference on Harmonization. Guideline on the validation of analytical procedures: definitions and terminology, Fed. Reg. 1995, 60(40), page 11260; Guideline on the validation of analytical procedures: methodology. Fed. Reg. 1997, 62(96), 27464). By simplifying the sample preparation chemistry involved in peptide mapping, the digest stability of the peptide product increases and further provides a benefit toward the automation of this protein analytical technique.

### SUMMARY OF THE INVENTION

The present invention relates to a method of high performance peptide mapping of a polypeptide with one or more cysteine residues using sulfitolysis. This method eliminates the need for sequential reduction, alkylation and quenching steps. Thus, the overall assay time is significantly reduced since the unfolding of the protein, reduction of disulfides and blocking of sulfhydryl groups are achieved in one simple step.

### BRIEF DESCRIPTION OF THE DRAWINGS

FIG. 1 depicts peptide mapping procedures for rhuMAb HER2.

FIG. 2 illustrates the sulfitolysis reaction mechanism.

FIG. 3 depicts tryptic peptide maps of rhuMAb HER2 obtained using reduction/alkylation (bottom) and sulfitolysis (top). Carboxymethyl cysteine-containing peptides are labeled, and the arrows indicate the elution position of the corresponding cysteine-S-sulfonate peptides. Carboxymethyl cysteine-containing peptides co-eluting with other non-cysteine peptides are identified in Table 1.

FIG. 4 depicts (a) Positive ion electrospray isonization (ESI) mass spectrum (MS) and (b) Matrix Assisted Laser Desorption/Ionization-Time of Flight (MALDI-TOF) mss spectrum (MS) of cys-S-sulfonate in VTITCR (T2L:, SEQ ID NO: 1) peptide.

FIG. 5 depicts ESI mass spectra obtained at different source conditions showing “In-source desulfonation of cys-S-sulfonate in NQVSLTCLVK (T36H, SEQ ID NO: 2)”.

FIG. 6 depicts reconstituted ion chromatograms (ESI-MS/MS) of (a) neutral loss of 80 Da, and (b) Neutral loss of 40 Da. The x-axis describes the retention time in minutes. The peptide assignment for each ion was made based upon its desulfonated mass.

FIGS. 7A-C depict time course of the sulfitolysis reaction at 37° C. Normalized peak areas of selected cys-S-sulfonate peptides were plotted as a function of reaction time. The peak eluting at approximately 30 minutes in the sulfitolysis map (see FIG. 2) was used as the internal standard for peak area normalization.

FIG. 8 depicts a detailed tryptic peptide map of rhuMAb HER2 to demonstrate the stability of methionine containing peptides under sulfitolysis conditions. Met-361-containing peptide (retention time 16 minutes) is not shown.

FIG. 9 depicts digest stability of the sulfitolysed rhuMAb tryptic peptide digests at ambient temperature.

### DETAILED DESCRIPTION OF THE PREFERRED EMBODIMENT

A. Preparation of Proteins for Peptide Mapping

In most generic terms, peptide mapping refers to the generation of peptides from a protein by partial hydrolysis, followed by separation and analysis of the fragments obtained. In order to generate a peptide map, partial hydrolysis of the starting protein to small peptides is needed, without complete hydrolysis that would yield amino acids. However, as noted before, for most proteins partial hydrolysis need to be preceded by unfolding of the protein, reduction of the disulfide bonds and blocking the sulihydryl groups so that efficient hydrolysis can occur.

Unfolding

Methods for unfolding proteins are well known in the art. Unfolding is performed using reagents known to denature proteins, such as, for example, guanidine hydrochloride, guanidine thiocyanate, or urea, although other denaturants can also be used. Such denaturants and their solutions, e.g. 4-6M guanidine hydrochloride and 6-8M urea, preferably, 6M guanidine hydrochloride and 8M urea, are commercially available or can be readily made, and are described in the example below. The denaturing reagent prevents refolding and crossfolding of the same protein molecule onto itself or two separate protein molecules onto each other. The only limitation is that the denaturant should not be so drastic as to cause damage to the protein that hinders subsequent analysis by peptide mapping.

Sulfitolysis

According to the traditional approach, unfolding of the protein is usually followed by the reduction of disulfide bonds (typically with dithiothreitol (DTT)) and the blocking of sulihydryl groups (typically with iodoacetic acid (IAA)) in separate steps. In contrast, the present invention provides a method in which unfolding is combined with oxidative sulfitolysis, as a one-step disulfide-reduction and sulfhydryl blocking reaction, providing a much simplified method for preparing a protein for peptide mapping, without compromising the quality of peptide mapping itself.

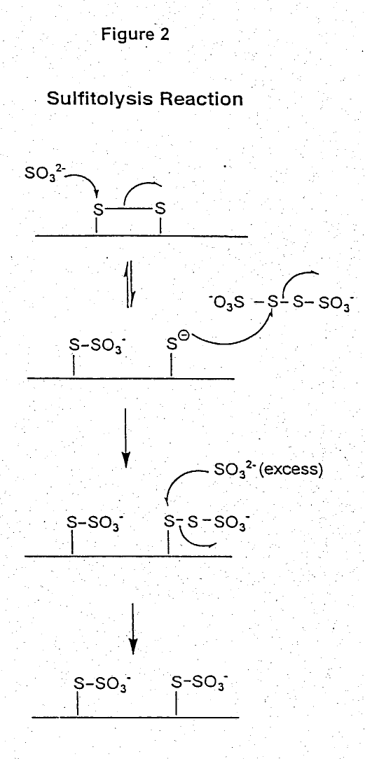

As illustrated in FIG. 2, the sulfitolysis reaction used in the methods of the present invention results in the formation of cysteine-S-sulfonate (cys-S-sulfonate) groups, which remain bound to and protect the cysteines during subsequent hydrolysis, e.g. enzymatic digestion of the protein. Conditions for converting cysteines to S-sulfonates by sulfitolysis are well known in the art, and are described in standard textbooks of chemistry. See also, the reaction scheme shown in FIG. 2, (Lundell and Schreitmuiller, 1999), and other references cited above.

In a particular embodiment, sulfitolysis of a protein to be prepared for peptide mapping is performed under alkaline conditions using sodium sulfite and sodium tetrathionate as a fast one-step disulfide reduction and thiol blocking reaction. These reagents may be added to the denaturing (unfolding) solution such that denaturing and sulfitolysis take place simultaneously, in one reaction step.

The time necessary to unfold the polypeptide, break the disulfide bonds and convert all cysteines to cysteine-S-sulfonates varies, and depends on variables like the identity of protein, the ;number of cysteines present, the denaturing solution, reaction temperature, etc. The term necessary and sufficient is used interchangeably whenever referred to in the context of subjecting the polypeptide to sulfitolysis. Typically, using traditional unfolding conditions, the conversion time at 37° C. varies between about 1 and 120 minutes, usually between about 1 and 60 minutes, more typically between about 1 and 30 minutes, preferably between about 1 and 10 minutes, most preferably between about 3 and 5 minutes. In a preferred embodiment of the present invention, combined unfolding and sulfitolysis is completed in about 3 to 5 minutes at 37° C.

The sulfitolysis of cysteines in accordance with the present invention is highly selective. For example, as shown in the example below, it has been experimentally found that the methionine residues present in the protein to be treated are not susceptible to sulfonation under sulfitolysis conditions used herein.

Furthermore, the cysteine-S-sulfonate groups formed are stable under standard peptide mapping conditions, which will be discussed in greater detail below. In particular, the cysteine-S-sulfonate groups are stable in the pH 1-9 range, at ambient temperature. If the conditions are different, for example during amino acid analysis, or Edman N-terminal sequence analysis, potential complications resulting from the instability of cysteine-S-sulfonate groups can be avoided by converting these groups into other, more stable groups, such as corresponding cys-S-alkyl derivatives.

Hydrolysis

Partial hydrolysis of proteins is commonly accomplished by using proteases, which provide high specificity and reproducibility. Proteolytic degradation of these cysteine-S-sulfonated polypeptides can employ a variety of different proteases in the methods of the present invention. Different proteases can be used alone or in combination in order to generate overlapping peptide fragments. Some commonly used proteases include chymotrypsin, elastase, ficin, papain, pepsin, thermolysin, thrombin and trypsin, to name a few. The substrate specificities of these and similar enzymes are well known, and the enzymes are commercially available. Some proteases are active in sodium dodecyl sulfate (SDS) such as chymotrypsin, elastase, trypsin and plasmin, which might be important in choosing the procedure used to separate the proteolytic digests (e.g. Cleveland method using SDS-PAGE).

To generate peptide maps, chemical hydrolysis techniques can be also used. Although chemical hydrolysis is seldom used exclusively, it often complements enzymatic methods in the process of generating overlapping fragments. Typically, chemical hydrolysis targets unusual amino acids, or unusual amino acid pairings. For example, Asp-Pro bonds can be hydrolyzed under mild acidic conditions, Asn-Gly bonds can be hydrolyzed with hydroxylamine, etc.

Separation of Proteolytic or Chemical Digests

It is necessary to separate the proteolytic and/or chemical digests of the present invention. Various separation techniques can be used as embodiments of the methods of the present invention. Such separation techniques include, without limitation, liquid chromatographic columns, capillary electrophoresis, resolution by one-dimensional SDS-PAGE analysis (Cleveland method), two dimensional separation on thin layer plates, as well as other known separation methods involved in peptide mapping.

Liquid chromatographic columns include micro high performance liquid chromatographic columns, for example, reverse-phase, ion-exchange, and affinity columns. Certain embodiments of the present invention may employ high performance liquid chromatographic columns, such as reverse-phase HPLC. Other embodiments may use ion-exchange HPLC to separate the proteolytic digests. Other embodiments may use one-dimensional SDS-PAGE (Cleveland method) analysis and may further use gradient gels in order to separate the peptides. One common approach uses about a 5% to 15% gradient. As noted above, in other methods of the present invention, two dimensional separation on thin layer plates can be used. One example of two-dimensional separation uses isoelectric focusing (IEF) in conjunction with SDS-page as a two-dimensional gel. Another example uses two thin layer plates as a two-dimensional separation system. One example of this two dimensional system is the Hunter Thin Layer Peptide Mapping Electrophoresis (HTLE) System which uses two thin layer cellulose plates (Cooper et al. 1983; Chiu et aL 1998). In another embodiment of the present invention, capillary electrophoresis (CE) can be used to separate the digests of the present invention. One example is capillary zone electrophoresis (CZW). Further, this can be used in conjunction with HPLC in order to analyze the separated digests of the present invention (Rush et al. 1993).

In a preferred embodiment of the present invention, reverse phase HPLC is used to separate the proteolytic digests. This method is illustrated in the example below.

The embodiments of the present invention can use commercially available reagents and apparatus to perform the methods of the present invention. Examples of such reagents are given in the following example.

The following Example is not limiting and is used to further describe the present invention:

### EXAMPLE

Materials and Methods

Materials

Trastuzumab (rhuMAb HER2) was produced by a Chinese hamster ovary cell line that was transfected with genes encoding the humanized light and heavy chain sequences (Carter et al. 1992). Sodium sulfite, DTT and synthetic peptide Met-enkephalin-Gly-Leu (YGGFMRGL) (SEQ ID NO: 3) was purchased from Sigma (St. Louis, Mo.). Sodium tetrathionate was obtained from Aldrich (Milwaukee, Wis.). lodoacetic acid (IAA) was from Research Organics (Cleveland, Ohio). N-tosyl-L-phenylalanine chloromethylketone (TPCK)-treated trypsin was from Worthington Biochemical Co. (Freehold, N.J.). PD-10 columns (SephadexG25, 2.2×8 cm) were from Pharmacia Biotech (Piscataway, N.J.). All other chemicals were analytical reagent grade.

Sulfitolysis

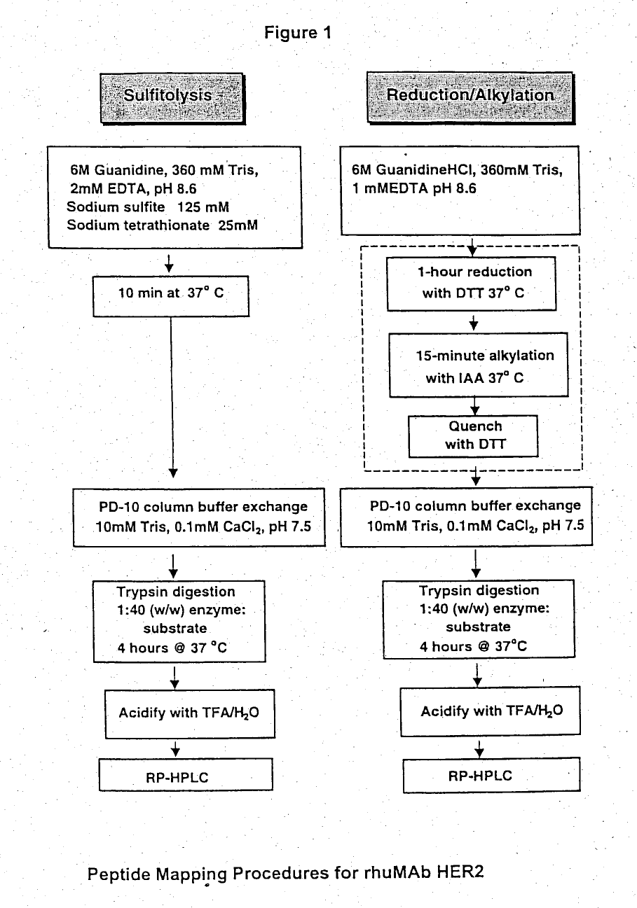

Lyophilized rhuMAb HER2 was reconstituted with purified water to a concentration of 25 mg/mL. A 1 mg (40 μL) aliquot of protein was combined with 960 μL of sulfitolysis reagent (6 M guanidine hydrochloride, 360 mM Tris, 2 mM EDTA, 125 mM sodium sulfite, 25 mM sodium tetrathionate, pH 8.6). The sample was incubated in a 37° C. water bath for 10 minutes. The sulfitolyzed sample was then desalted on a PD-10 column equilibrated with trypsin digest buffer (10 mM Tris, 0.1 mM CaCl₂, pH 7.5) and the buffer exchanged protein was collected in a final volume of 1.8 mL. TPCK-trypsin, dissolved in 1 mM HCl, was added at an enzyme : substrate ratio of 1:40 (w/w) and digestion was conducted at 37° C. for 4 hours. The reaction was stopped by adding 50 μL of 10% TFA. The procedure is summarized in FIG. 1.

Reduction and Alkylation

The rhuMAb HER2 sample (1 mg, 40 μl) was combined with 960 μL of 6 M guanidine hydrochloride, pH 8.6, and DTT was added to a final concentration of 10 mM. The sample was then incubated for 1 hour at 37° C. IAA was added (final concentration 35 mM) and the sample was incubated in the dark for 15 minutes at 37° C. and excess IAA was quenched by a second addition of DTT (final concentration 40 mM). The S-carboxymethylated protein was then desalted and digested with trypsin as described above.

Chromatography

Reversed-phase HPLC separation of tryptic peptide fragments was performed on a Hewlett Packard 1090L system with a Vydac C18 column (218TP54, 250×4.6 mm, 5 μM (Vydac, Hesperia, Calif.), operating at a flow rate of 1.0 mL/min. The column oven temperature was set at 37° C. Typically 65 μg (150 μL) was injected onto the column and a linear gradient of 0.1% TFA in water (solvent A) and 0.1% TFA in acetonitrile (solvent B) was used to elute the peptide fragments. The percentage of solvent B was varied from 0 to 40% B over 80 minutes and 40 to 60% B over 10 minutes, followed by an isocratic step of 60% B for 5 minutes. Peptides were detected by UV absorbance at 214 nm. The tryptic peptides were labeled starting with “T” followed by a number to show the order in which it is expected from the N-terminus and followed by either “L” for the light chain or “H” for the heavy chain. Thus T2L (SEQ ID NO: 1) refers to a second tryptic peptide from the N-terminus of the light chain.

Mass Spectral Analysis

The tryptic map obtained by the sulfitolysis method was analyzed for new peaks using on-line liquid chromatography/electrospray ionization-mass spectrometry (LC/ESI-MS). The conditions used for LC separation were the same as specified above. A Finnigan LCQ mass spectrometer with ESI source was used to acquire positive ion spectra of peptides. The full scan spectra (m/z 200-2000) and zoom scan spectra were obtained in a data dependent fashion. The mass spectrometer was set to the following parameters: ion spray voltage, 5 kV; capillary voltage, 30 V; capillary temperature, 250° C.; sheath gas and auxiliary gas flow were controlled at 80 psi and 20 psi, respectively. The neutral loss ESI/MS-MS experiments were performed on a Finnigan TSQ 7000 instrument with the voltages for the heated capillary and the tube lens at 40 and 30 respectively. These parameters were determined by using a Cys-S-sulfonate peptide T11L (SEQ ID NO: 19) for tuning the instrument with respect to the tube lens and heated capillary voltage in order to minimize in-source fragmentation, while tuning the collision offset voltage and the target Argon pressure for maximum cleavage in the collision cell.

Further improvements in sensitivity were obtained by opening up the parent and the daughter resolution to allow maximum ion transmission. Both the quadrupoles 1 and 3 (Q1 and Q3) were scanned with a mass offset of 80 Da for singly charged ions and 40 Da for doubly charged ions equal to the mass of the SO₃ neutral loss group. The Q2 was operated in the transmission mode with the collision gas on. As the Q1 scans, peptides of different masses are allowed into the collision cell Q2. However, only the peptides generating −80 (or −40) daughter ions are allowed to pass through Q3 to record the signal. Observed masses in the neutral loss mode tend to be slightly higher compared to regular ESI/MS, and this is attributed to the wider m/z window.

TABLE 1

| | | Expected | Observed Mass with Cys (SO₃H) | | | |
|---|---|---|---|---|---|---|
| Cys-S-Sulfonate | | Mass with | | | Neutral | Neutral |
| Containing Peptide* | Peptide Sequence | Cys (SO₃H) | MALDI-TOF/MS | ESI/MS | Loss 40 | Loss 80 |
| T19H (222-225) | SCDK^{¥§} | 531.6 | — | — | — | 530.6 |
| T20L (212-214) | GEC* | 387.1 | — | — | — | — |
| T15H Clip (202-213) | ICNVHKPSNTK^{§} | 1434.6 | — | 1434.2 | 1432.8 | — |
| T19-20L | SFNRGEC^{§} | 891.3 | 891.6 | 890.9 | — | 890.1 |
| T2L (19-24) | VTITCR | 771.4 | 771.4 | 770.9 | — | 772.5 |
| T11H (88-98) | AEDTAVYYCSR | 1356.4 | 1356.7 | 1356.1 | 1355.6 | 1356.9 |
| T17-18L (189-207) | HKVYACEVTHQGLSSPVTK^{§} | 2163.1 | 2163.6 | 2163.2 | 2161.2 | — |
| T18L (191-207) | VYACEVTHQGLSSPVTK | 1898.0 | 1898.4 | 1897.8 | 1895.6 | — |
| T2H (20-30) | LSCAASGFNIK^{§} | 1189.6 | 1189.8 | 1189.2 | 1189.0 | 1188.6 |
| T14H (137-150) | STSGGTAALGCLVK | 1343.7 | 1343.2 | 1343.2 | 1342.4 | 1342.6 |
| T36H (364-373) | NQVSLTCLVK | 1183.6 | 1183.5 | 1183.1 | 1184.8 | 1182.6 |
| T22H (259-277) | TPEVTCVVVDVSHEDPEVK^{§} | 2161.0 | 2161.1 | 2160.8 | 2157.0 | — |
| T7L Clip (77-92) | SLQPEDFATYYCQQHY^{§} | 2071.8 | — | 2071.2 | — | — |
| T41H (420-442) | WQQGNVFSCSVMHESALHNHYTQK^{§} | 2823.2 | — | 2823.8 | 2822.4 | — |
| T7L Clip (77-103) | SLQPEDFATYYCQQHYTPPTFGQGTK^{§} | 3187.4 | — | 3187.1 | — | — |
| T7L (67-103) | SGTDFTLTISSLQPEDFATYYCQQHYTPPTFGQGTK^{§} | 4209.9 | 4211.1 | 4210.7 | — | — |
| T20H (226-251) | THTCPPCPAPELLGGPSVFLFPPKPK | 2889.4 | 2889.1 | — | 2887.6 | 2889.0 |
| T11L (127-142) | SGTASVVCLLNNFYPR | 1819.9 | 1819.9 | 1819.8 | 1820.0 | — |

*Dipeptide CK (H324-325) is eluted in the void.
^{¥}Identified by Edman sequence analysis.
^{§}Co-elutes with other non-cyesteine containing peptides.

Fractions of the new peaks shown in the tryptic map using the sulfitolysis method were also collected and subjected to MALDI-TOF/MS analysis in the linear mode. All of the analyses were performed on a Voyager Elite-DE instrument (PerSeptive Biosystems, Framingham, Mass.) using 2,4,6-trihydroxyacetophenone (THAP) as the UV-absorbing matrix, as described in detail, for example, by Papac et al. (Anal. Chem, 1996, pages 3215-3223).

Results and Discussion

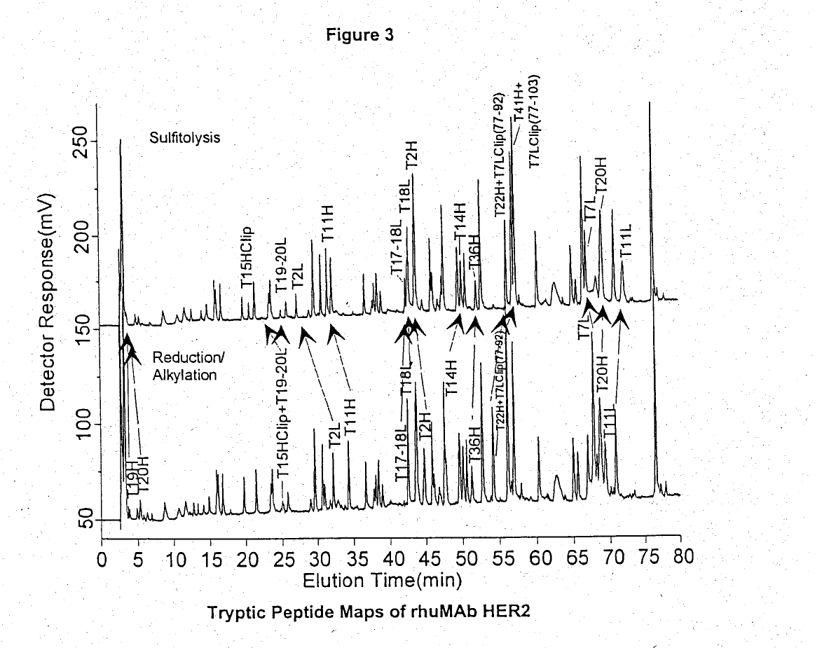

The rhuMAb HER2 tryptic peptide map with reduction/alkylation was previously optimized and validated in order to demonstrate specificity, precision and robustness, using the general approach described in Kannan et al. (J. Pharm. Biomed. Anal. 1997. vol. 16, page 631). Guanidine hydrochloride (6 M) was the preferred protein-unfolding reagent for rhuMAb HER2; the use of 8 M urea was found to give incomplete alkylation. A trypsin-to-antibody ratio of 1:40 (w/w) and a digestion time of 4 hours at 37° C. were optimal for completion of digestion with minimal non-tryptic cleavage. The chromatography conditions were optimized to maximize resolution while keeping the run time to about 90 minutes. The resulting peptide map was characterized using MALDI-TOF/MS and N-terminal Edman sequencing to identify the tryptic peptides (R. Harris & F. Shen, unpublished data). The locations of the 15 S-carboxymethyl cysteine-containing peptides in the peptide map are shown in FIG. 3.

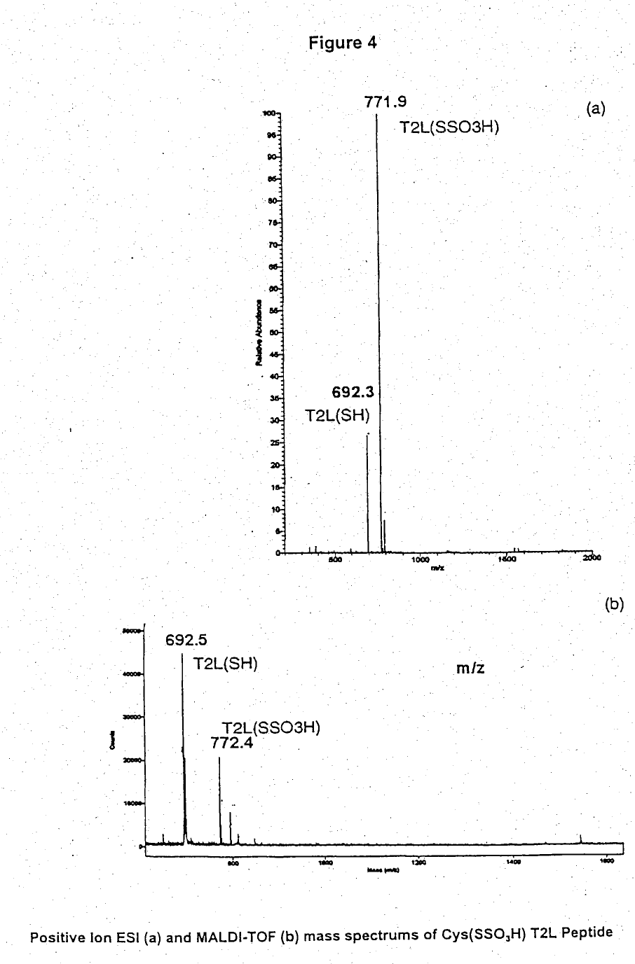

For the sulfitolysis procedure, the protein unfolding reagent, trypsin digestion and chromatography conditions were the same as for the reduction/alkylation method; the only difference was in the sulfitolysis step. The peptide map obtained using sulfitolysis had a peptide pattern similar to that obtained with reduction/alkylation, except that the cysteine-S-sulfonate-containing peptides had altered retention behavior (FIG. 3). Table 1 shows these peptides which were identified using electrospray ionization and MALDI-TOF/MS. A typical ESI mass spectrum is shown in FIG. 4(a) for the T2L peptide (Cys-S-sulfonate VTITCR, SEQ ID NO: 1). The protonated molecular ion was observed at a m/z of 771.9 with an excellent signal-to-noise ratio. An additional signal with an m/z of 692.3, which is approximately 80 Da less than the main signal, corresponds to the desulfonated T2L (SEQ ID NO: 1) peptide. The desulfonation (due to the breakage of the S-SO₃⁻ bond) is facile compared to breakage of the peptide bonds and readily occurs in the source region of the electrospray. Smith and Zhou (Smith et al. Methods Enzymol. 1990. vol. 193, page 374) have described the facile reduction of an intermolecular disulfide bond in egg white Lysozyme peptide during mass analysis. Further evidence for desulfonation during the mass spectral ionization process is obtained from the MALDI-TOF spectrum. In the spectrum of the T2L (SEQ ID NO: 1) peptide, the desulfonated species was the predominant ion observed at a m/z of 692.5. The protonated Cys-S-sulfonate molecular ion (m/z 772.4) and its sodium and potassium adducts were also observed (FIG. 4(b)). The presence of higher amounts of the desulfonated species in the MALDI-TOF spectrum is probably due to the higher activation energy involved in MALDI process compared to the electrospray process. Patterson and Katta (Patterson et al. Anal. Chem. 1994. vol. 66, page 3727) have reported fragmentation of the disulfide-linked peptides into their reduced forms during the analysis of an Asp-N digest of a recombinant hematopoietic growth factor by MALDI-MS.

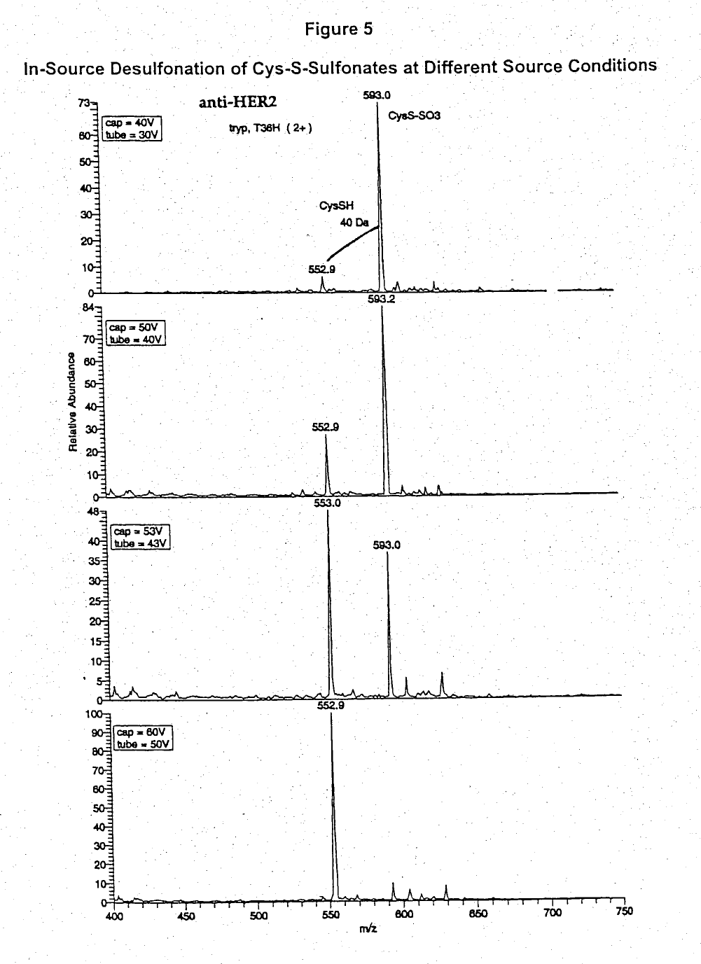

The heated capillary and the tube lens voltage modulations can manipulate the recovery of sulfonated and desulfonated peptide forms. The peptide fragment T36H (NQVSLTCLVK) (SEQ ID NO: 2) showed mostly doubly charged S-sulfonated ion, when the voltages for the heated capillary and the tube lens were 40 and 30 respectively. However, only desulfonated ion of T36H (SEQ ID NO: 2) was obtained when both the voltages were raised by 20 (FIG. 5). The observation of the thiol-containing peptide may also suggest that both the free thiol and the Cys-S-sulfonate may be present due to an incomplete sulfitolysis reaction (FIG. 2).

In order to confirm the absence of any free thiol-containing peptides, an experiment was performed in which the sulfitolysis reaction mixture was further treated with IAA (58 mM) for 15 minutes at 37° C. followed by buffer exchange and trypsin digestion in the usual manner. The tryptic peptide map of rhuMAb HER2 treated in this fashion was identical to the regular sulfitolysis map. No new peaks attributable to the cys-S-carboxymethyl derivatives were observed, indicating the absence of free thiol-containing peptides and the completion of the sulfitolysis reaction.

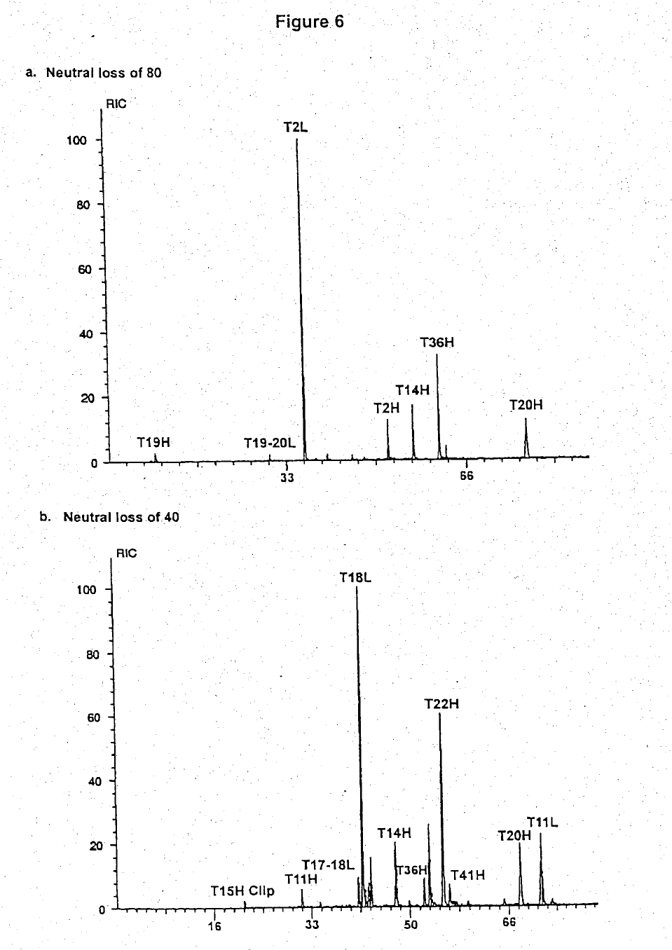

The electrospray-induced fragmentation of the SO₃ group (loss of 80 amu) was used as a tool for the selective identification of cys-S-sulfonate-containing peptides in the rhuMAb HER2 peptide map. The neutral loss ESI-MS-MS spectra of the rhuMAb HER2 peptide map are shown in FIG. 6. Using this technique, the inventor has identified all except one of the cys-containing peptides. The T7L (SEQ If) NO: 5) peptide and its clipped form were not detected in either of the neutral loss modes. These peptides primarily exist at higher charge states (+3 and +4). However, preliminary data indicates detection of the peptides using higher order neutral loss experiments with mass offsets at −27 and −20. The sulfitolysis helps in the separation of the cys-S-sulfonate containing peptides from the co-eluting peptides in a peptide map. The neutral loss technique for the sulfitolysed protein digest is also a convenient method for identification of cys-containing peptides in a complex peptide map without the interference of other peptides. One can get a peptide map of cysteine containing peptides.

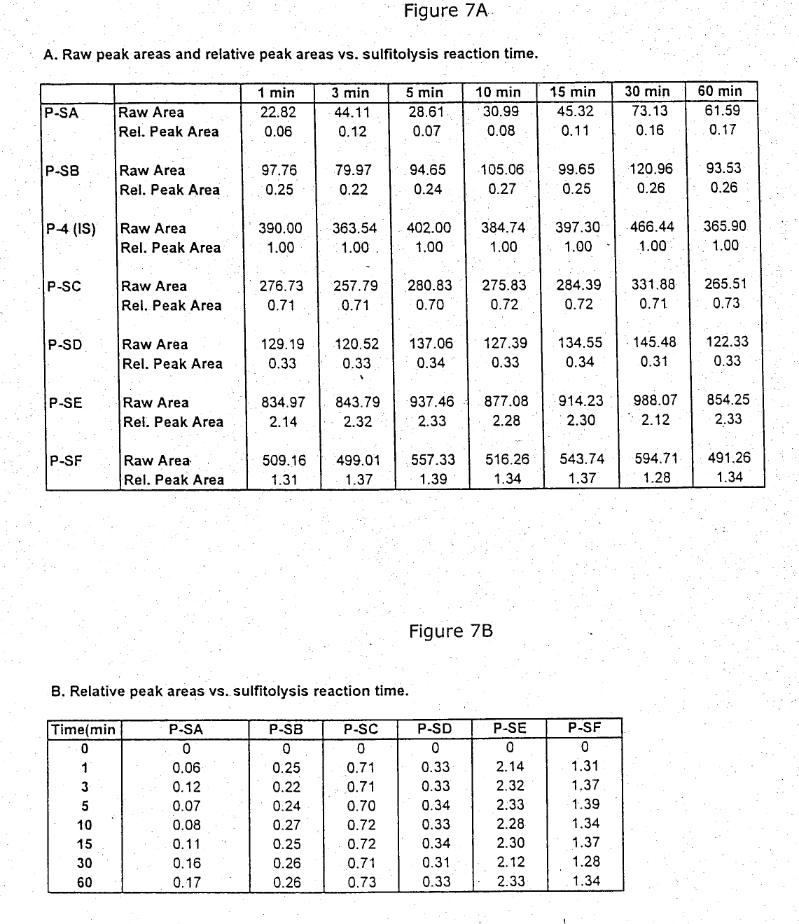

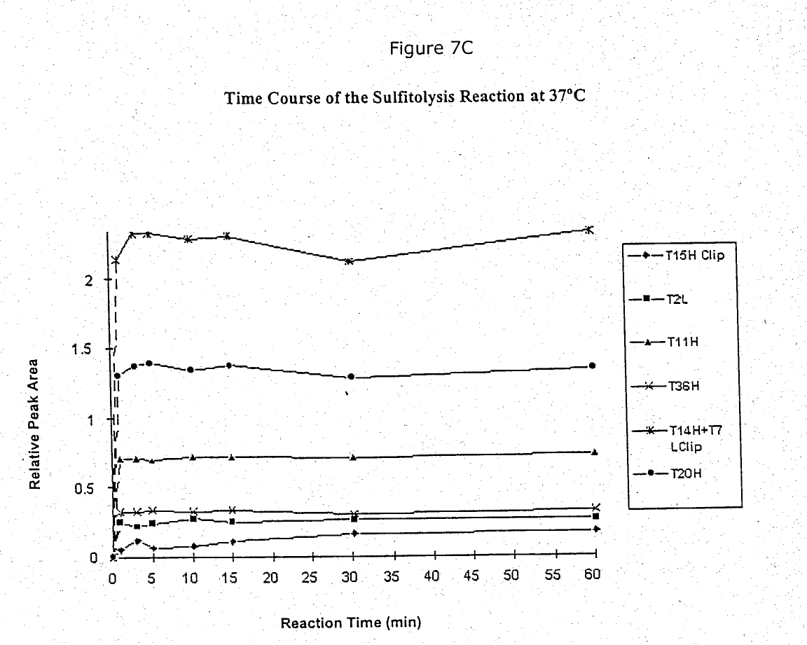

The extent of the sulfitolysis reaction was monitored by generating a series of maps with increasing sulfitolysis time from 1-60 minutes at 37° C. Lundell and Schreitmuller (Lundell et al. Analytical Biochemistry, 1999, vol. 266, page 31) reported that at least 2h might be required to convert all the cysteines to thiosulfates during sulfitolysis. However, in the inventor's hands a sulfitolysis time of 3-5 minutes seems to be sufficient for a complete reaction. The rapid reaction kinetics is illustrated in FIG. 7C. The raw peak areas and relative peak areas of sulfitolysis reaction are described in FIGS. 7A-7B. The relative peak areas of six cys-S-sulfonate-containing peptides, selected over the entire chromatogram based on their absence of co-eluting with other peptides, reach a plateau within 5-10 minutes. Patrick and Lagu (Patrick et al. Anal. Chem. 1992, vol. 64, page 507) also obtained comparable results under similar reaction conditions with their proinsulin-fusion protein; complete sulfitolysis was obtained in about 30 minutes at ambient temperature. In comparison the optimal DTT-reduction and IAA-alkylation times for rhuMAb HER2 are 60 and 15 minutes (at 37° C.), respectively.

Another benefit of sulfitolysis over reduction/alkylation is the stability of the sulfitolysis reagent. The peptide map obtained with 2-week old sulfitolysis reagent (stored at 2-8° C.) is comparable to that obtained with the fresh reagent (data not shown). In contrast, the DTT solution has to be freshly prepared or frozen in single use aliquots whereas the IAA has to be freshly prepared and protected from light during storage and reaction. The stability of the sulfitolysis reagent will be a significant advantage for automation of the peptide map procedure.

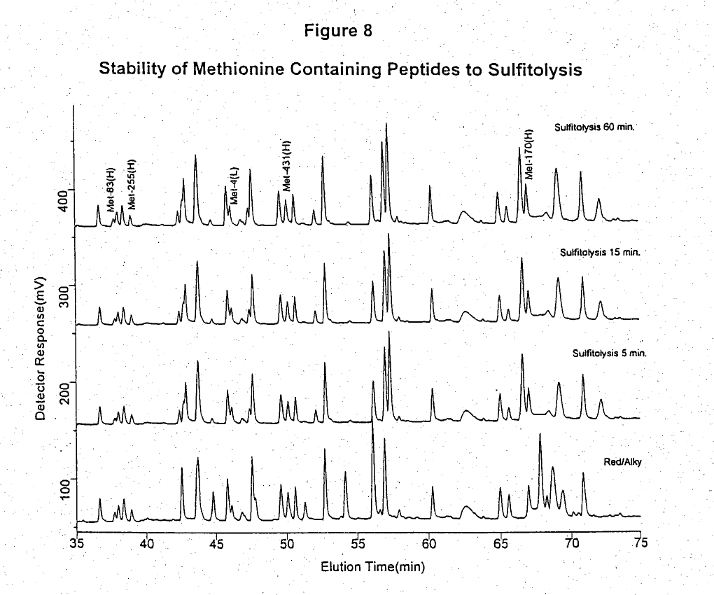

An undesirable side reaction of carboxymethylation with IAA is the alkylation of methionine residue (Hirs. Methods Enzymol. 1967, vol. 11, page 199). Jones et al. (Analytical Biochemistry. 1994, vol. 216, page 135) have obtained a reproducible peptide map by optimizing reduction and alkylation steps to minimize methionine alkylation while maintaining efficient conversion of cysteine to S-carboxymethyl cysteine. The inventor has investigated whether the methionine residues are susceptible to sulfonation under sulfitolysis conditions by monitoring the methionine containing peptides in the tryptic map of rhuMAb HER2 as a function of increasing sulfitolysis time. No significant reduction of peak area of methionine-containing peptides was observed, even at 60 minutes at 37° C. (FIG. 8). Of the six methionine residues in the rhuMAb HER2 molecule, Met-255 and Met-431 are reported to be solvent accessible and hence more reactive (Shen et al. Tech. Protein Chem. 1996, VIII, page 275). In the present study, the sulfitolysis is performed in the presence of 6M guanidine hydrochloride, where the antibody is presumed to be fully unfolded and therefore all the methionine residues are expected to be equally accessible to the sulfitolysis reagent.

Met-enkephalin synthetic peptide was used to further probe the reactivity of methionine residues towards the sulfitolysis reagent. Reversed-phase HPLC analysis of the Met-enkephalin-sulfitolysis reaction mixture indicated no evidence of met-sulfonate formation. The only discernible reaction of met-enkephalin was formation of Met (O)-enkephalin at a level of <1%, which is most likely due to dissolved oxygen in the reaction buffer.

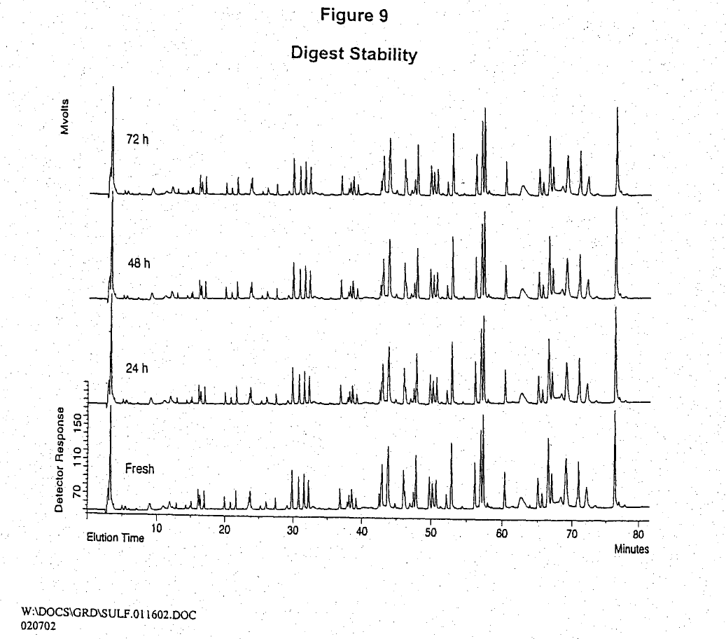

Since the Cys-S-sulfonate group is potentially unstable under certain conditions (Cole. Methods Enzymol. 1967. vol. 11, page 206) the inventor has investigated the stability of the peptide digest for storage at pH 2 and various temperatures. The TFA-acidified digest (˜pH 2) was found to be stable for at least 72 hours at ambient temperature (FIG. 9), and for at least 2 weeks at 2-8° C. and −70° C. (data not shown). These data indicate that Cys-S-sulfonate group is stable under peptide mapping conditions and the present data is in agreement with reported stability in the range of pH 1-9 at ambient temperature (Greene et al. Protective Groups in Organic Synthesis. 2^{nd} ed. John Wiley & Sons, Inc., New York. 1991; Chan. Biochemistry 1968. vol. 7, page 4247). The Cys-S-sulfonate group is cleaved to Cys-SH in the presence of 6M hydrochloric acid and neat TFA at elevated temperatures, conditions observed during amino acid analysis and Edman N-terminal sequence analysis, respectively. During the sequential Edman degradation we have observed a blank cycle at Cys-S-sulfonate residue, reduction in yield of the PTH-amino acid following the Cys-S-sulfonate and an increased lag of the N+1 residue. This observation is consistent with reversible S to N rearrangement of the sulfonyl group (Reed Harris, personal communication). An alternate mechanism involving the reaction of the sulfonyl group with the thio urea group of the phenylthiocarbamyl (PTC)-peptide is also possible (Milligan et al. J. Chem. Soc. 1962. page 2172). In order to avoid these potential complications during N-terminal sequencing the Cys-S-sulfonate peptide could be converted to the corresponding Cys-SH peptide by reacting with the reducing agent tris (carboxyethyl) phosphine and alkylated with IAA or 4-vinyl pyridine to give a stable derivative.

The precision of the sulfitolysis peptide map assay was demonstrated for chromatography repeatability and assay repeatability. The standard deviation (SD) for the mean retention time of selected marker peaks was 0.01 minutes and the relative standard deviation (RSD) for their relative peak areas ranged from 1.5-4.1%. Both the SD of the retention times and the RSD of the relative peak areas for the sulfitolysis peptide map are similar to those for the reduction/alkylation method, and are comparable to other complex peptide maps (Kannan et al., J. Pharm. Biomed. Anal. 1997. vol. 16, page 631; Carlson et al. Analytica Chimica Acta. 1997. vol. 352, page 221). This method satisfies the requirements of the ICH guidelines (International Conference on Harmonization. Guideline on the validation of analytical procedures: definitions and terminology, Fed. Reg. 1995, 60(40), page 11260; Guideline on the validation of analytical procedures: methodology. Fed. Reg. 1997, 62(96), 27464).

Conclusion

The Example described above is set forth solely to assist in the understanding of the invention. Thus, those skilled in the art will appreciate that the methods of the present invention can provide a method of peptide mapping of a polypeptide comprising one or more cysteine residues.

One skilled in the art would readily appreciate that the present invention is well adapted to carry out the objects and obtain the ends and advantages mentioned, as well as those inherent therein. The methods and procedures described herein are presently representative of preferred embodiments and are exemplary and are not intended as limitations on the scope of the invention. Changes therein and other uses will occur to those skilled in the art which are encompassed within the spirit of the invention.

It will be readily apparent to one skilled in the art that varying substitutions and modifications may be made to the invention disclosed herein without departing from the scope and spirit of the invention.

All patents and publications mentioned in the specification are indicative of the levels of those skilled in the art to which the invention pertains. All patents and publications are herein incorporated by reference to the same extent as if each individual publication was specifically and individually indicated to be incorporated by reference.

The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions indicates the exclusion of equivalents of the features shown and described or portions thereof. It is recognized that various modifications are possible within the scope of the invention. Thus, it should be understood that although the present invention has been specifically disclosed by preferred embodiments and optional features, modification and variation of the concepts herein disclosed may be resorted to by those skilled in the art, and that such modifications and variations are considered to be falling within the scope of the invention, which is limited only by the following claims.

### REFERENCES

1. Hancock, W. S. New Methods in Peptide Mapping for Characterization of Proteins, CRC Press, New York, 1995

2. Garnick, R. L. Dev. Biol. Stand. 1992, 76, 117.

3. Hoff, E. R.; Chloupek, R. C. Methods Enzymol. 1996, 271, 51.

4. Dougherty Jr., J. J.; Snyder, L. M.; Sinclair, R. L.; Robins, R. H. Anal. Biochem. 1990, 190, 7.

5. Kannan, K.; Mulkerrin, M. G.; Zhang, M.; Gray, R., Steinharter, T.; Sewerin, K.; Baffi, R.; Harris, R.; Karunatilake, C; J. Pharm. Biomed. Anal. 1997, 16, 631.

6. Nadler, T.; Blackburn, C.; Mark, J., Gordon, N.; Regnier, F. E.; Vella, G. J. Chromatogr. A. 1996, 743, 91.

7. Heath, W. F.; Belagaje, R. M.; Brooke, G. S.; Chance, R. E.; Hoffman, J. A.; Long, H. B.; Reams, S. G.; Roundtree, C.; Shaw, W. N.; Slieker, L. J.; et al. J. Biol. Chem. 1992, 267, 419.

8. Belagaje, R. M.; Reams, S. G.; Ly, S. C.; Prouty, W. F. Protein Science 1997, 6, 1953.

9. Cartwright, T.; Keckwick, R. G. O. Biochim. Biophys. Acta. 1971, 236, 550.

10. Novotny, J.; Franek, F.; Sorn, F. Eur. J. Biochem. 1970, 16, 278.

11. Milburn, P. J.; Scheraga, H. A. J. Protein Chem. 1988,7,377.

11. Carter, P.; Presta, L.; Gorman, C. M.; Ridgway, J. B. B.; Henner, D.; Wong, W. L.; Rowland, A. M.; Kotts, C.; Carver, M. E.; Shepard, H. M. Proc. Natl. Acad. Sci. USA 1992, 89, 4285.

12. Cohen, R. L. Cancer Biotherapy & Radiopharmaceuticals. 1999, 14, 1.

13. Carter, P.; Fendly, B. M.; Lewis, G. D.; Sliwkowski, M. X. Breast Disease 2000, 11, 103.

14. Parekh, R. B. Biologicals 1994, 22, 113.

15. Rao, P.; Williams, A.; Baldwin-Ferro, A.; Hanigan, E.; Kroon, D.; Makowskji, M.; Meyer, E.; Numuswan, V.; Rubin, E.; Tran, A. BioPharm 1991, 4, 38.

16. Harris, R. J.; Murnane, A. A.; Utter, S. L.; Wagner, K. L.; Cox, E. T.; Polastri, G. D.; Helder, J. C.; Sliwkowski, M. B. Bio/Technology 1993, 11, 1293.

17. Harris, R. J. J. Chromatogr. A 1995, 705, 129.

18. Papac, D. I.; Wong, A.; Jones, A. J. S. Anal. Chem. 1996, 68, 3215.

19. Smith, D. L.; Zhou, Z. Methods Enzymol. 1990, 193, 374.

20. Patterson, S. D.; Katta, V. Anal. Chem. 1994, 66, 3727

21. Lundell, N.; Schreitmuiller, T. Analytical Biochemistry, 1999, 266, 31.

22. Patrick, J. S.; Lagu, A.; Anal. Chem. 1992, 64, 507.

23. Hirs, C. H. W. Methods Enzymol. 1967, 11, 199.

24. Jones, M. D.; Merewether, L. A.; Clogston, C. L.; Lu, H. S. Analytical Biochemistry 1994,216,135.

25. Shen, F.; Kwong, M. Y.; Keck, R. G.; Harris, R. J. Tech. Protein Chem. 1996, VIII, 275.

26. Cole, R. D. Methods in Enzymology. 1967, 11, 206.

27. Greene, T. W.; Wuts, P. G. M. Protective Groups in Organic Synthesis, 2nd edition. John Wiley & Sons, Inc., New York 1991.

28. Chan, W. W. C. Biochemistry, 1968, 7, 4247.

29. Milligan, B.; J. M. Swan, J. Chem. Soc., 1962, 2172.

30. Carlson, A. D.; Hofer, J. D.; Riggin, R. M. Analytica Chimica Acta 1997, 352, 221.

31. International Conference on Harmonization. Guideline on the validation of analytical procedures: definitions and terminology, Fed. Reg. 1995, 60(40), 11260.

32. International Conference on Harmonization. Guideline on the validation of analytical procedures: methodology. Fed. Reg. 1997, 62(96), 27464.

33. Cooper, J. A.; Sefton, B. M.; Hunter, T. M. Methods Enzymol. 1983, 99, 387.

34. Chiu, R.; Boule, W. J.; Meek, J.; Smeal, T.; Hunter, T.; Karin, M. Cell 1988, 54, 541.

35. Rush, R. S.; Derby, P. L.; Strickland, T. W.; Rohde, M. F. Anal. Chem. 1993, 65, 1834.

What is claimed is:

1. A method of preparing a polypeptide comprising one or more cysteine residues for peptide mapping, comprising subjecting said polypeptide to sulfitolysis under denaturing conditions for a time sufficient to convert said cysteine residues into cysteine-S-sulfonates.

2. The method of claim 1 wherein sulfitolysis is performed using sodium sulfite and sodium tetrathionate.

3. The method of claim 2 wherein sulfitolysis is performed at about 37° C.

4. The method of claim 2 wherein sulfitolysis is performed for about 1 to 120 minutes.

5. The method of claim 2 wherein sulfitolysis is performed for about 1 to about 60 minutes.

6. The method of claim 2 wherein sulfitolysis is performed for about 3 to 5 minutes.

7. The method of claim 1 wherein denaturing conditions are provided by the presence of a denaturant selected from the group consisting of guanidine hydrochloride, guanidine thiocyanate, and urea.

8. The method of claim 7 wherein denaturing conditions are provided by the presence of 4-6M guanidine hydrochloride.

9. The method of claim 7 wherein denaturing conditions are provided by the presence of 6-8M urea.

10. A method of peptide mapping of a polypeptide comprising one or more cysteine residues, comprising the steps of:
(a) subjecting the polypeptide to sulfitolysis for a time sufficient to convert said cysteine residues into cysteine-S-sulfonates,
(b) hydrolyzing the cysteine-S-sulfonated polypeptide to provide peptide fragments of said polypeptide; and
(c) separating the peptide fragments produced in step (b).

11. The method of claim 10 wherein sulfitolysis is performed using sodium sulfite and sodium tetrathionate.

12. The method of claim 11 wherein sulfitolysis is performed at about 37° C.

13. The method of claim 10 wherein sulfitolysis is performed for about 1 to 120 minutes.

14. The method of claim 10 wherein sulfitolysis is performed for about 1 to 60 minutes.

15. The method of claim 10 wherein sulfitolysis is performed for about 3 to 5 minutes.

16. The method of claim 10 wherein hydrolysis is performed by using a proteolytic enzyme.

17. The method of claim 10 wherein hydrolysis is performed by chemical hydrolysis.

18. The method of claim 16 wherein said proteolytic enzyme is selected from the group consisting of chymotrypsin, elastase, ficin, papain, pepsin, thermolysin, thrombin, trypsin, and plasmin.

19. The method of claim 10 wherein said peptide fragments are separated by high performance liquid chromatography (HPLC).

20. The method of claim 10 wherein said peptide fragments are separated by reverse-phase HPLC.

21. The method of claim 10 wherein said peptide fragments are separated by ion-exchange HPLC.

22. The method of claim 10 wherein said peptide fragments are separated by resolution on one-dimensional SDS-PAGE analysis.

23. The method of claim 10 wherein said peptide fragments are separated by capillary electrophoresis.

24. The method of claim 10 wherein said peptide fragments are separated by two dimensional separation on thin layer plates.


SEQUENCE LISTING (19 sequences)

SEQ ID NO: 1 - protein, 6 aa, homo sapiens
VTITCR

SEQ ID NO: 2 - protein, 10 aa, homo sapiens
NQVSLTCLVK

SEQ ID NO: 3 - protein, 8 aa, homo sapiens
YGGFMRGL

SEQ ID NO: 4 - protein, 11 aa, homo sapiens
AEDTAVYYCSR

SEQ ID NO: 5 - protein, 36 aa, homo sapiens
SGTDFTLTISSLQPEDFATYYCQQHYTPPTFGQGTK

SEQ ID NO: 6 - protein, 16 aa, homo sapiens
SLQPEDFATYYCQQHY

SEQ ID NO: 7 - protein, 16 aa, homo sapiens
SLQPEDFATYYCQQHY

SEQ ID NO: 8 - protein, 4 aa, homo sapiens
SCDK

SEQ ID NO: 9 - protein, 3 aa, homo sapiens
GEC

SEQ ID NO: 10 - protein, 12 aa, homo sapiens
ICNVNHKPSNTK

SEQ ID NO: 11 - protein, 7 aa, homo sapiens
SFNRGEC

SEQ ID NO: 12 - protein, 19 aa, homo sapiens
HKVYACEVTHQGLSSPVTK

SEQ ID NO: 13 - protein, 17 aa, homo sapiens
VYACEVTHQGLSSPVTK

SEQ ID NO: 14 - protein, 11 aa, homo sapiens
LSCAASGFNIK

SEQ ID NO: 15 - protein, 14 aa, homo sapiens
STSGGTAALGCLVK

SEQ ID NO: 16 - protein, 19 aa, homo sapiens
TPEVTCVVVDVSHEDPEVK

SEQ ID NO: 17 - protein, 23 aa, homo sapiens
WQQGNVFSCSVMHEALHNHYTQK

SEQ ID NO: 18 - protein, 26 aa, homo sapiens
THTCPPCPAPELLGGPSVFLFPPKPK

SEQ ID NO: 19 - protein, 16 aa, homo sapiens
SGTASVVCLLNNFYPR